(19) United States
(12) Patent Application Publication
(10) Pub. No.: US 2018/0116922 A1
(43) Pub. Date: May 3, 2018

(54) Absorbent Liquid Skin Cleanser

(71) Applicant: Barry Earl Chandler, Houston, TX (US)

(72) Inventor: Barry Earl Chandler, Houston, TX (US)

(21) Appl. No.: 15/796,330

(22) Filed: Oct. 27, 2017

Related U.S. Application Data
(60) Provisional application No. 62/413,546, filed on Oct. 27, 2016.

(51) Int. Cl. A61K 8/26; A61K 8/73; A61K 8/02; A61Q 19/10; A61K 8/9794; A61K 8/9789

(52) U.S. Cl. CPC A61K 8/26; A61K 8/731; A61K 8/0279; A61K 2800/48; A61K 8/9794; A61K 8/9789; A61Q 19/10

(57) ABSTRACT

An absorbent liquid skin cleanser includes a quantity of hollow cellulose particles, a quantity of absorbent minerals, and a quantity of surfactants to remove oil, dirt, and grease from the skin of a user through absorption. The quantity of hollow cellulose particles, the quantity of absorbent minerals, and the quantity of surfactants are suspended in a quantity of water as a transfer medium. The viscosity of the quantity of water is increased through mixing a quantity of thickening compound. The increased viscosity allows the user to effectively lather the absorbent liquid skin cleanser on the user's skin to remove most contaminants.

The current application claims a priority to the U.S. Provisional Patent application Ser. No. 62/413,546 filed on Oct. 27, 2016.

### FIELD OF THE INVENTION

The present invention relates generally to a liquid skin cleaner. More specifically, the present invention relates to an absorbent skin cleaner, that implements hollow cellulose particles and absorbing minerals, which is applied to the skin of the user to clean the skin.

### BACKGROUND OF THE INVENTION

Numerous types of cleaners have been devised to effectively clean skin. Most cleaners designed for cleaning skin are found in a liquid form. Some liquid cleaners have an abrasive substance suspended within the fluid medium use hard or solid particles such as pumice, plastic, or walnut shells or other solid abrasives to help clean. These hard particles remove the soil from the skin by an abrasive action. The hard or solid particle, such as pumice, plastic, peach pits, walnut shells in skin cleaners abrade the skin when used in the skin cleaning process.

The present invention is an absorbent liquid skin cleanser that includes hollow cellulose particles of various sizes to remove oil, dirt, and grease through absorption. The selection of cellulose plant fibers used in the skin cleaner absorb different size and types of oil grease, dirt and bacteria. The quantity/volume and type of the cellulose, hollow plant fibers are sufficient to do the majority of the skin cleaning. Three or more hollow

The surfactants coat the inside and outside of the hollow plant cellulose. In the skin cleaning process, the hollow plant cellulose does the majority of the cleaning. The surfactants aid in holding the dirt, oil, grease and bacteria in suspension within the hollow plant cellulose as not to adhere to the skin, allowing these contaminates to be washed down the drain during rinsing.

The absorbing nature of the present invention has been tested for the removal of bacteria and the absorption of oils that create odor, allowing the present invention to remove odors such as fish, onion, garlic, and body odor from skin. Whereas hollow, cellulose plant fibers gently exfoliate skin while absorbing the dirt, oil, grease and bacteria. This absorbing process along with the conditioners and gentle surfactants leave the skin soft and smooth as if lotion was used.

### DETAIL DESCRIPTIONS OF THE INVENTION

The present invention is an absorbent liquid skin cleanser to remove contaminants from a user's skin, such as dirt, debris, oils, grease, and bacteria. The present invention exfoliates the skin and absorbs the contaminants. The contaminants are absorbed by the present invention and then flushed away from the user's skin using water.

The present invention comprises a quantity of hollow cellulose particles, a quantity of absorbent minerals, a quantity of surfactants, a quantity of water, and a quantity of thickening compound. The quantity of hollow cellulose particles exfoliates the skin and absorbs contaminants deposited on the skin. The hollow cellulose particles are preferably corncob particles from 100 to 300 microns. Corncob particles are preferred due to lightweight, absorbent, and abrasive properties. The quantity of absorbent minerals additionally absorbs contaminants, similar to the quantity of hollow cellulose particles. The quantity of absorbent minerals is preferred to be bentonite; however, the absorbent minerals may also be zeolite or any other absorbent microporous solid that is able to absorb oils and grease. The quantity of surfactants emulsifies oils and greases to be washed away from the skin with water. The quantity of water and the quantity of thickening compound provide a medium for the quantity of hollow cellulose particles, the quantity of absorbent minerals, and the quantity of surfactants to be suspended within, in order to efficiently apply the present invention to the user's skin. The quantity of thickening compound increases the viscosity of the present invention such that the present invention may be lathered effectively on the user's skin. The quantity of hollow cellulose particles, the quantity of absorbent minerals the quantity of surfactants, the quantity of water, and the quantity of thickening compound are heterogeneously mixed into a cleansing mixture.

TABLE 1
Absorbent Liquid Skin Cleanser

| Component | Approximate wt. % |
|---|---|
| Hollow Cellulose Particles | 5 |
| Absorbent Minerals | 1 |
| Surfactants | 5-6 |
| Water | 70-80 |
| Thickening Compound | <5 |

In accordance to Table 1, the quantity of hollow cellulose particles is approximately 5% by weight (wt. %) of the cleansing mixture. This fraction of the hollow cellulose particles within the cleansing mixture is sufficient to be evenly distributed throughout the cleansing mixture, as well as, to bind with any debris, oils, or grease on the user's skin effectively. The quantity of absorbent minerals is approximately 1 wt. % of the cleansing mixture, such that the quantity of absorbent minerals is sufficient to absorb oils and grease on the user's skin. The quantity of surfactants is between 5 wt. % and 6 wt. % of the cleansing mixture, where the quantity of surfactants is sufficient to remove any dirt and debris from the user's skin in conjunction with the hollow cellulose particles. The quantity of water is between 70 wt. % and 80 wt. % of the cleansing mixture. This ratio of water provides an effective medium to suspend the quantity of hollow cellulose particles, the quantity of absorbent minerals, and the quantity of surfactants and to distribute the present invention on the user's skin. The quantity of thickening compound is less than 5 wt. % of the cleansing mixture, in order to increase viscosity of the present invention greater than the viscosity of water, which allows the present invention to lather effectively on the user's skin.

TABLE 2
Absorbent Liquid Skin Cleanser

| Component | Approximate wt. % |
|---|---|
| Hollow Cellulose Particles | 5 |
| Absorbent Minerals | 1 |
| Surfactants | 5-6 |
| Water | 70-80 |
| Thickening Compound | <5 |
| Conditioning Agents | 2 |
| Fragrance Compounds | <1 |

In accordance to the preferred embodiment of the present invention, the present invention comprises a quantity of conditioning agents, as shown in Table 2. The quantity of conditioning agents is conducive to maintain the condition of skin. The quantity of conditioning agents is heterogeneously mixed into the cleansing mixture. The quantity of conditioning agents is less than 2 wt. % of the cleansing mixture, in order to be distributed throughout the cleansing mixture to be effective when the present invention is lathered onto the user's skin. The quantity of conditioning agents is selected from a group consisting of aloe vera, birch bark extracts, and combinations thereof. Aloe vera and birch bark extracts are preferred as these compounds condition the skin imparting anti-inflammatory and softening properties to the user's skin.

In accordance to some embodiments of the present invention, the present invention comprises a quantity of fragrance compounds, exemplified in Table 2. The quantity of fragrance compounds appeal to a user's sense of smell. The quantity of fragrance compounds is mixed into the cleansing mixture, in order to impart a fragrance to the present invention. The quantity of fragrance compounds is preferred to be less than 1 wt. % of the cleansing mixture, such that the quantity of fragrance compounds is able to diffuse from the user's skin after being deposited on the user's skin during a wash cycle.

TABLE 3
Effectiveness of Absorbent Liquid Skin Cleanser on Microorganisms

| Test | Contact Time | CFU/Carrier | | Percent |
|---|---|---|---|---|
| Microorganism | (seconds) | Initial | Final | Reduction |
| S. aureus | 20 | 3.70e+06 | 3.43e+03 | 99.9073% |
| P. aeruginosa | 20 | 9.0e+05 | 2.60e+04 | 97.3737% |

Staphylococcus aureus (S. aureus) and Pseudomonas aeruginosa (P. aeruginosa) are two anti-biotic resistant bacterial pathogens. S. aureus is a leading cause of hospital-acquired infections; commonly a wound infection after surgery. P. aeruginosa is found on and in medical equipment causing cross-infections in hospitals and clinics. In accordance to Table 3, the present invention is effective at dealing with both pathogens. After twenty seconds of exposure, the present invention has been tested to reduce the colony forming unit (CFU) to carrier ratio for each bacterium. The ratio of CFU to carrier denotes the estimated number of cells present based on the ability to give rise to colonies on the carrier medium. The present invention reduced the CFU to carrier ratio by 99.9073% for S. aureus and 97.3737% for P. aeruginosa after 20 seconds of contact.

Although the invention has been explained in relation to its preferred embodiment, it is to be understood that many other possible modifications and variations can be made without departing from the spirit and scope of the invention as hereinafter claimed.

What is claimed is:

1. An absorbent liquid skin cleanser comprises:
a quantity of hollow cellulose particles;
a quantity of absorbent minerals;
a quantity of surfactants;
a quantity of water;
a quantity of thickening compound;
the quantity of hollow cellulose particles, the quantity of absorbent minerals, the quantity of surfactants, the quantity of water, and the quantity of thickening compound being heterogeneously mixed into a cleansing mixture;

2. The absorbent liquid skin cleanser, as claimed in claim 1, comprises:
the quantity of hollow cellulose particles being approximately 5% by weight (wt. %) of the cleansing mixture;
the quantity of absorbent minerals being approximately 1 wt. % of the cleansing mixture;
the quantity of surfactants being between 5 wt. % and 6 wt. % of the cleansing mixture;
the quantity of water being between 70 wt. % and 80 wt. % of the cleansing mixture;
the quantity of thickening compound being less than 5 wt. % of the cleansing mixture;

3. The absorbent liquid skin cleanser, as claimed in claim 1, comprises:
a quantity of conditioning agents;
the quantity of conditioning agents being heterogeneously mixed into the cleansing mixture;

4. The absorbent liquid skin cleanser, as claimed in claim 3, comprises:
the quantity of conditioning agents being less than 2 wt. % of the cleansing mixture;

5. The absorbent liquid skin cleanser, as claimed in claim 3, wherein the quantity of conditioning agents is selected from the group consisting of aloe vera, birch bark extracts, and combinations thereof;

6. The absorbent liquid skin cleanser, as claimed in claim 1, comprises:
a quantity of fragrance compounds;
the quantity of fragrance compounds being mixed into the cleansing mixture;

7. The absorbent liquid skin cleanser, as claimed in claim 6, comprises:
the quantity of fragrance compounds being less than 1 wt. % of the cleansing mixture.

8. The absorbent liquid skin cleanser, as claimed in claim 1, wherein the quantity of absorbents minerals is bentonite.

9. The absorbent liquid skin cleanser, as claimed in claim 1, wherein the quantity of hollow cellulose particles is corncob particles.

10. The absorbent liquid skin cleanser, as claimed in claim 9, wherein the corncob particles have a mean diameter from 100 to 300 microns.

11. An absorbent liquid skin cleanser comprises:
a quantity of hollow cellulose particles;
a quantity of absorbent minerals;
a quantity of surfactants;
a quantity of water;
a quantity of thickening compound;
a quantity of conditioning agents;
the quantity of hollow cellulose particles, the quantity of absorbent minerals, the quantity of surfactants, the quantity of water, the quantity of thickening compound, and the quantity of conditioning agents being heterogeneously mixed into a cleansing mixture;

12. The absorbent liquid skin cleanser, as claimed in claim 11, comprises:
the quantity of hollow cellulose particles being approximately 5% by weight (wt. %) of the cleansing mixture;
the quantity of absorbent minerals being approximately 1 wt. % of the cleansing mixture;
the quantity of surfactants being between 5 wt. % and 6 wt. % of the cleansing mixture;
the quantity of water being between 70 wt. % and 80 wt. % of the cleansing mixture;
the quantity of thickening compound being less than 5 wt. % of the cleansing mixture;

13. The absorbent liquid skin cleanser, as claimed in claim 11, comprises:
the quantity of conditioning agents being less than 2 wt. % of the cleansing mixture;

14. The absorbent liquid skin cleanser, as claimed in claim 11, wherein the quantity of conditioning agents is selected from the group consisting of aloe vera, birch bark extracts, and combinations thereof;

15. The absorbent liquid skin cleanser, as claimed in claim 11, comprises:
a quantity of fragrance compounds;
the quantity of fragrance compounds being mixed into the cleansing mixture;

16. The absorbent liquid skin cleanser, as claimed in claim 15, comprises:
the quantity of fragrance compounds being less than 1 wt. % of the cleansing mixture.

17. The absorbent liquid skin cleanser, as claimed in claim 11, wherein the quantity of absorbents minerals is bentonite.

18. The absorbent liquid skin cleanser, as claimed in claim 11, wherein the quantity of hollow cellulose particles is corncob particles.

19. The absorbent liquid skin cleanser, as claimed in claim 18, wherein the corncob particles have a mean diameter from 100 to 300 microns.
